(19) United States
(12) Patent Application Publication
(10) Pub. No.: US 2015/0245820 A1
(43) Pub. Date: Sep. 3, 2015

(54) ULTRASONIC MEASUREMENT APPARATUS AND ULTRASONIC MEASUREMENT METHOD

(71) Applicant: SEIKO EPSON CORPORATION, Tokyo (JP)

(72) Inventor: Natsumi TAMADA, Shiojiri-shi (JP)

(21) Appl. No.: 14/625,645

(22) Filed: Feb. 19, 2015

(30) Foreign Application Priority Data
Feb. 28, 2014 (JP) 2014-038977

(51) Int. Cl. A61B 8/08

(52) U.S. Cl. CPC A61B 8/5223; A61B 8/085

(57) ABSTRACT

A scanning line immediately above a blood vessel is detected using a received signal of a reflected wave of an ultrasonic wave transmitted to the blood vessel, and candidates for front and rear walls of the blood vessel are detected based on the received signal of the scanning line. Then, vascular front and rear walls pairs of front and rear walls are narrowed down from the candidates, and the narrowed-down vascular front and rear walls pair is regarded as one blood vessel and artery/vein identification is performed for each blood vessel. Measurement of vascular function information is performed for the blood vessel determined to be an artery. Determination of an artery/vein is performed based on the relative relationship between the contraction time and the expansion time of the blood vessel.

### BACKGROUND

1. Technical Field

The present invention relates to an ultrasonic measurement apparatus that performs measurement using an ultrasonic wave.

2. Related Art

As an example of measuring biological information with an ultrasonic measurement apparatus, the evaluation of a vascular function or the determination of a vascular disease is performed. For example, the intima media thickness (IMT) of the carotid artery, which is an indicator of arteriosclerosis, is measured. In the measurement relevant to the IMT or the like, it is necessary to locate the carotid artery and appropriately determine the measurement point. Typically, the operator places an ultrasonic probe on the neck, locates the carotid artery to be measured while watching a B-mode image displayed on the monitor, and manually sets the found carotid artery as a measurement point.

Although skill is required in order to execute such a series of measurement operations quickly and locate the carotid artery appropriately in the related art, a function to assist the measurement operation has been devised in recent years. For example, JP-A-2008-173177 discloses a method of detecting the vascular wall automatically using the strength of a reflected wave signal from the body tissue, which is obtained by processing the amplitude information of the received reflected wave, and the moving speed of the body tissue, which is obtained by processing the phase information of the received reflected wave. Specifically, a boundary between the vascular wall and the blood flow region is detected based on the first finding that the strength of the reflected wave signal in the blood flow region in the blood vessel is very small compared with the strength of the reflected wave signal in the vascular wall and the second finding that the moving speed calculated from the phase information of the reflected wave signal is high in the blood flow region and low in the vascular wall.

However, in the detection method disclosed in JP-A-2008-173177, a blood vessel can be detected, but it is not possible to determine whether the blood vessel is an artery or a vein. In general, the artery exhibits pulsation, but the vein does not exhibit pulsation. For this reason, the operator tends to simply think that the artery and the vein can be identified by the presence or absence of pulsation. However, in blood vessels relatively close to the heart, such as the internal jugular vein, even veins may exhibit pulsation due to the pressure of the right atrium being transmitted thereto. Therefore, it is difficult to perform correct identification from only the presence or absence of pulsation.

### SUMMARY

An advantage of some aspects of the invention is to implement an ultrasonic measurement technique for identifying an artery and a vein.

A first aspect of the invention is directed to an ultrasonic measurement apparatus including: a transmission and reception control unit that controls transmission of an ultrasonic wave to a blood vessel and reception of a reflected wave; a contraction and expansion time calculation unit that calculates a contraction time and an expansion time of the blood vessel based on a received signal of the reflected wave; and a type determination unit that determines a type of the blood vessel using a relative relationship between the contraction time and the expansion time.

As another aspect of the invention, the first aspect of the invention may be configured as an ultrasonic measurement method including: controlling transmission of an ultrasonic wave to a blood vessel and reception of a reflected wave; calculating a contraction time and an expansion time of the blood vessel based on a received signal of the reflected wave; and determining a type of the blood vessel using the relative relationship between the contraction time and the expansion time.

According to the first aspect and the like of the invention, the type of the blood vessel can be determined using the relative relationship between the contraction time and the expansion time of the blood vessel. That is, even in the case of a vein with pulsation, such as an internal jugular vein, it is possible to appropriately determine the type of the blood vessel by identifying the artery and the vein.

As a second aspect of the invention, the ultrasonic measurement apparatus according to the first aspect of the invention may be configured such that the type determination unit determines the type of the blood vessel using a ratio between the contraction time and the expansion time.

According to the second aspect of the invention, it is possible to determine the type of the blood vessel using the contraction time and the expansion time of the blood vessel. An artery and a vein have a characteristic that the degree of change in the blood vessel diameter at the time of expansion of the artery is largely different from the degree of change in the blood vessel diameter at the time of expansion of the vein. That is, since a large difference occurs in the expansion time, it is possible to determine the type of the blood vessel based on the ratio between the expansion time and the contraction time of the blood vessel.

As a third aspect of the invention, the ultrasonic measurement apparatus according to the first or second aspect of the invention may be configured such that the type determination unit determines an artery and a vein as the type of the blood vessel.

According to the third aspect of the invention, it is possible to determine an artery and a vein as the type of the blood vessel.

As a fourth aspect of the invention, the ultrasonic measurement apparatus according to any one of the first to third aspects of the invention may be configured such that the type determination unit determines that the blood vessel is an artery using at least a value that a ratio between the contraction time and the expansion time can have when the blood vessel is an artery.

According to the fourth aspect of the invention, it is possible to determine that the blood vessel is an artery.

As a fifth aspect of the invention, the ultrasonic measurement apparatus according to any one of the first to fourth aspects of the invention may be configured such that the type determination unit determines that the blood vessel is a vein using at least a value that a ratio between the contraction time and the expansion time can have when the blood vessel is a vein.

According to the fifth aspect of the invention, it is possible to determine that the blood vessel is a vein.

As a sixth aspect of the invention, the ultrasonic measurement apparatus according to any one of the first to fifth aspects of the invention may be configured such that the ultrasonic measurement apparatus further includes a front and rear walls detection unit that detects a front wall and a rear wall of the blood vessel using the received signal of the reflected wave, and the contraction and expansion time calculation unit calculates the contraction time and the expansion time by determining a systole and a diastole of the blood vessel from a temporal change in the front and rear walls.

According to the sixth aspect of the invention, the contraction time and the expansion time are calculated by determining a systole and a diastole of the blood vessel from a temporal change in the front and rear walls of the blood vessel.

As a seventh aspect of the invention, the ultrasonic measurement apparatus according to any one of the first to sixth aspects of the invention may be configured such that the contraction and expansion time calculation unit calculates the contraction time and the expansion time using the received signal of a period of at least one cardiac beat.

According to the seventh aspect of the invention, the contraction time and the expansion time are calculated using the received signal of a period of at least one cardiac beat. A blood vessel repeats expansion and contraction with a period of one cardiac beat as a unit. Therefore, it is possible to determine the type of the blood vessel correctly if the contraction time and the expansion time in a period of at least one cardiac beat can be calculated.

### BRIEF DESCRIPTION OF THE DRAWINGS

The invention will be described with reference to the accompanying drawings, wherein like numbers reference like elements.

FIG. 1 is a diagram showing the system configuration of an ultrasonic measurement apparatus.

FIG. 2 is a flowchart of the main process performed by the ultrasonic measurement apparatus.

FIG. 3 is an explanatory diagram of ultrasonic measurement.

FIGS. 4A to 4C are diagrams showing an example of a received signal of a reflected wave of an ultrasonic signal.

FIGS. 5A and 5B are explanatory diagrams of detection of scanning lines immediately above the blood vessel.

FIGS. 6A to 6C are explanatory diagrams of narrowing down of vascular front and rear walls pairs.

FIGS. 7A and 7B are diagrams showing examples of the waveform of a change in the blood vessel diameter.

FIGS. 8A to 8D are diagrams showing examples of the waveform of a change in the blood vessel diameter and the waveform of a diameter change rate.

FIGS. 9A and 9B are diagrams showing an example of the expansion contraction time ratio.

FIG. 10 is a diagram showing the functional configuration of the ultrasonic measurement apparatus.

FIG. 11 is a diagram showing the configuration of a storage unit.

FIG. 12 is a diagram showing the data structure of vascular front and rear walls pair data.

FIG. 13 is a flowchart illustrating the flow of the process of detecting the scanning lines immediately above the blood vessel.

FIG. 14 is a flowchart illustrating the flow of the process of detecting the vessel wall depth position candidate.

FIG. 15 is a flowchart illustrating the process of narrowing down vascular front and rear walls pairs.

FIG. 16 is a flowchart illustrating the flow of the artery determination process.

### DESCRIPTION OF EXEMPLARY EMBODIMENTS

### Overall configuration

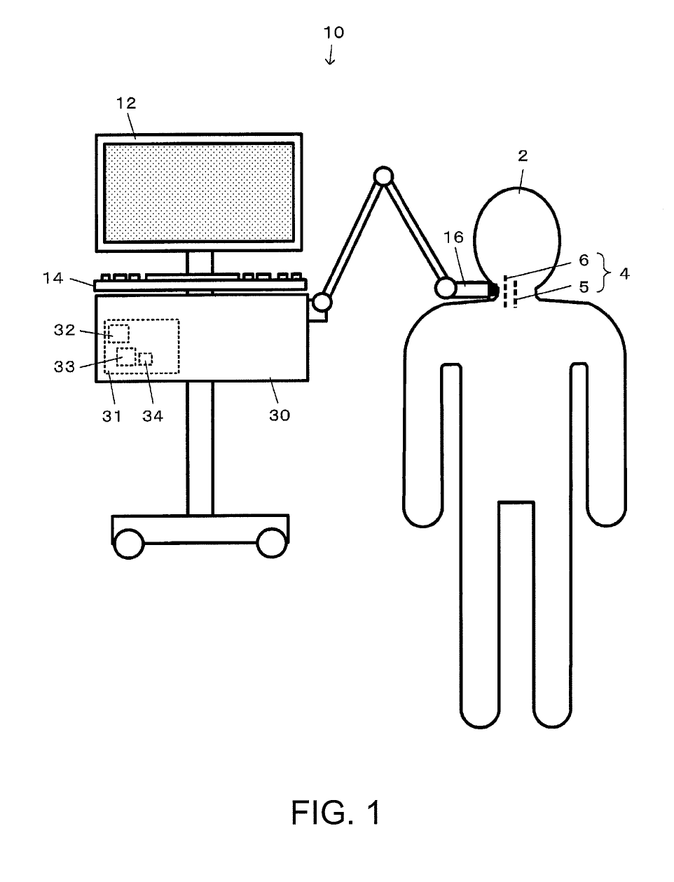

FIG. 1 is a diagram showing an example of the system configuration of an ultrasonic measurement apparatus 10 according to the present embodiment. The ultrasonic measurement apparatus 10 is an apparatus that measures biological information of a subject 2 using an ultrasonic wave. In the present embodiment, an artery 5 and a vein 6 of blood vessels 4 are automatically identified, and vascular function information, such as the intima media thickness (IMT) of the artery 5, is measured as a piece of biological information. Needless to say, it is also possible to measure other vascular function information, such as a blood vessel diameter or blood pressure measured from the blood vessel diameter, in addition to the IMT.

The ultrasonic measurement apparatus 10 includes a touch panel 12, a keyboard 14, an ultrasonic probe 16, and a processing device 30. A control board 31 is mounted in the processing device 30, and is connected to each unit of the apparatus, such as the touch panel 12, the keyboard 14, and the ultrasonic probe 16, so that signal transmission and reception therebetween are possible.

Not only various integrated circuits, such as a central processing unit (CPU) and an application specific integrated circuit (ASIC), but also a storage medium 33, such as an IC memory or a hard disk, and a communication IC 34 for realizing data communication with an external device are mounted on the control board 31. The processing device 30 realizes various functions according to the present embodiment, such as identification of the artery 5 and the vein 6, measurement of vascular function information for the identified artery 5, and image display control of the measurement result, including ultrasonic measurement by executing a control program stored in the storage medium 33 with the CPU 32 or the like.

Specifically, by the control of the processing device 30, the ultrasonic measurement apparatus 10 transmits and emits an ultrasonic beam from the ultrasonic probe 16 to the subject 2 and receives the reflected wave. Then, by performing amplification and signal processing on a received signal of the reflected wave, it is possible to generate reflected wave data, such as a temporal change or position information of a structure in the body of the subject 2. Images of respective modes of so-called A mode, B mode, M mode, and color Doppler are included in the reflected wave data. Measurement using an ultrasonic wave is repeatedly performed at predetermined periods. The measurement unit is referred to as a “frame”.

By setting a region of interest (tracking point) in the reflected wave data as a reference, the ultrasonic measurement apparatus 10 can perform so-called “tracking” that is tracking each region of interest between different frames and calculating the displacement.

### Overview

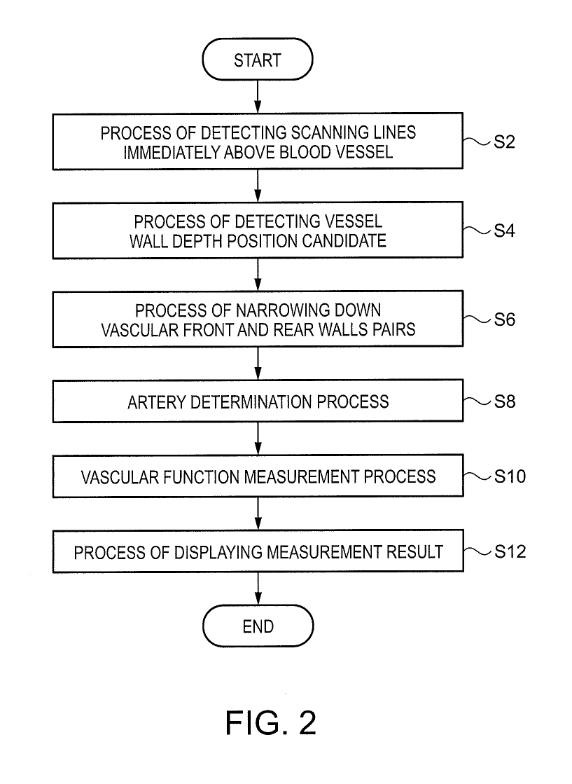

First, the overview of the process leading up to the measurement of vascular function information will be described. FIG. 2 is a flowchart showing the flow of the main process performed by the ultrasonic measurement apparatus 10. It is assumed that the ultrasonic probe 16 is directed toward the carotid artery of the subject 2 by the operator.

First, the ultrasonic measurement apparatus 10 detects an ultrasonic transducer (can also be a scanning line) located immediately above the blood vessel regardless of the distinction of arteries and veins (step S2). This is referred to as a “scanning line immediately above the blood vessel”. In addition, “immediately above” referred to herein, needless to say, includes a position directly above the blood vessel center literally, but also has the meaning allowing a slight shift in a radial direction from the position immediately above in a range that is sufficient to measure the vascular function information of interest. In addition, “immediately above” or “directly above” is not necessarily the meaning of a vertically upward direction (opposite direction to gravity), but is the meaning in the operation of the operator who handles the ultrasonic probe 16 to place the ultrasonic probe 16 “immediately above” or “directly above” the blood vessel on the body surface (meaning in a manual).

Then, a candidate at a depth position that seems to be a vascular wall is detected from the reflected wave data in the scanning lines immediately above the blood vessel (step S4). Although a part regarded as the front wall (vascular wall facing the skin side) of the blood vessel or the rear wall (vascular wall located opposite the front wall) of the blood vessel is detected in this stage, a body part other than the blood vessels may be included in depth position candidates since the part has not yet been determined as a blood vessel. Therefore, the ultrasonic measurement apparatus 10 narrows down the pairs of front and rear walls of the blood vessels from the detected depth position candidates (step S6). The narrowed-down pair of depth position candidates is called a “vascular front and rear walls pair”.

Then, the ultrasonic measurement apparatus 10 performs artery determination for each narrowed-down vascular front and rear walls pair, thereby identifying whether or not the vascular front and rear walls pair corresponds to an artery (step S8). Then, vascular function measurement is performed for the vascular front and rear walls pair determined to be the artery 5 (step S10), and the measurement result is displayed on the touch panel 12 (step S12). The content of the vascular function measurement may be other content without being limited to the IMT, and a known technique can be appropriately used.

### Principle

Next, each step will be described in detail. First, a step of detecting the scanning lines immediately above the blood vessel (step S2 in FIG. 2) will be described. The detection of the scanning lines immediately above the blood vessel is based on the movement of body tissues. That is, a blood vessel position is determined based on the finding that blood vessels move largely periodically with the beating of the heart but the movement of other body tissues around the blood vessels is small compared with the movement of the blood vessels.

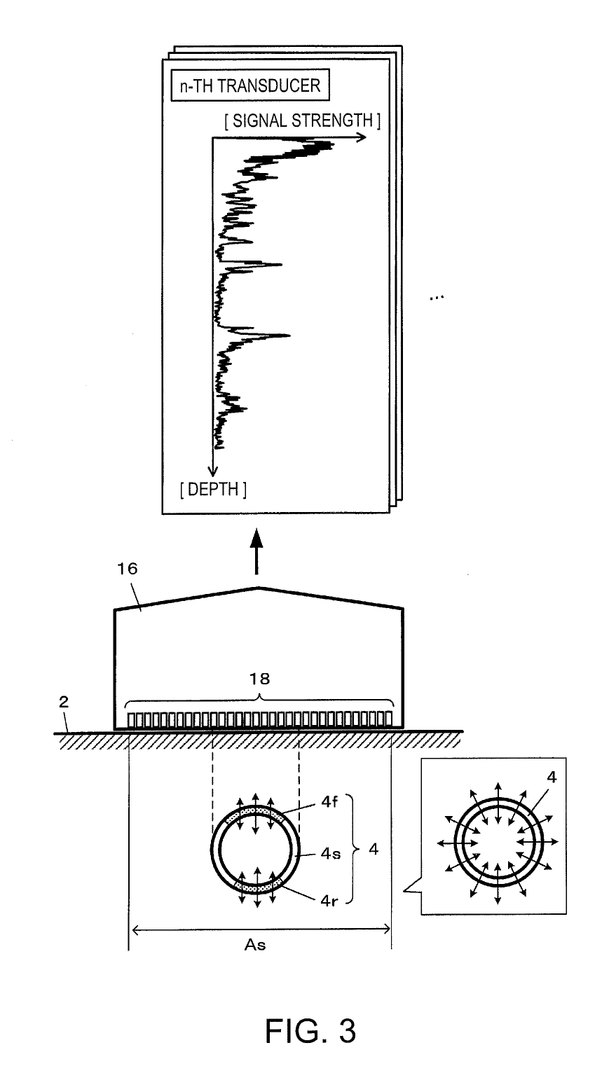

FIG. 3 is a diagram schematically showing a state where the ultrasonic probe 16 is in contact with the body surface of the subject 2 in order to perform ultrasonic measurement, and is a diagram showing the cross-section of the blood vessel 4 in a short-axis direction. A plurality of ultrasonic transducers 18 are built into the ultrasonic probe 16. In the example shown in FIG. 3, one ultrasonic beam is emitted from each ultrasonic transducer 18 toward the bottom from the top in the diagram. The range covered by the ultrasonic transducer 18 is a probe scanning range As. The ultrasonic transducers 18 may also be provided in a plurality of columns in a depth direction in the diagram, that is, may be provided in a planar shape. Alternatively, the ultrasonic transducers 18 may be provided only in a horizontal direction in only one column in the depth direction in the diagram.

The blood vessel 4 repeats approximately isotropic expansion/contraction due to the beating (expansion/contraction) of the heart. Therefore, a stronger reflected wave can be received as the area of the surface perpendicular to the direction of the ultrasonic beam becomes larger. However, it becomes more difficult to receive the reflected wave as the direction of the reflected wave becomes parallel to the beam direction. For this reason, in the ultrasonic measurement, the reflected wave from a front wall 4f and a rear wall 4r of the blood vessel 4 is detected strongly, but the reflected wave from a lateral wall 4s is weak. In other words, if there is the blood vessel 4 in the probe scanning range As, a strong reflected wave relevant to the front wall 4f and the rear wall 4r appears in the reflected wave signal at the position of the ultrasonic transducer 18 located immediately above the blood vessel 4.

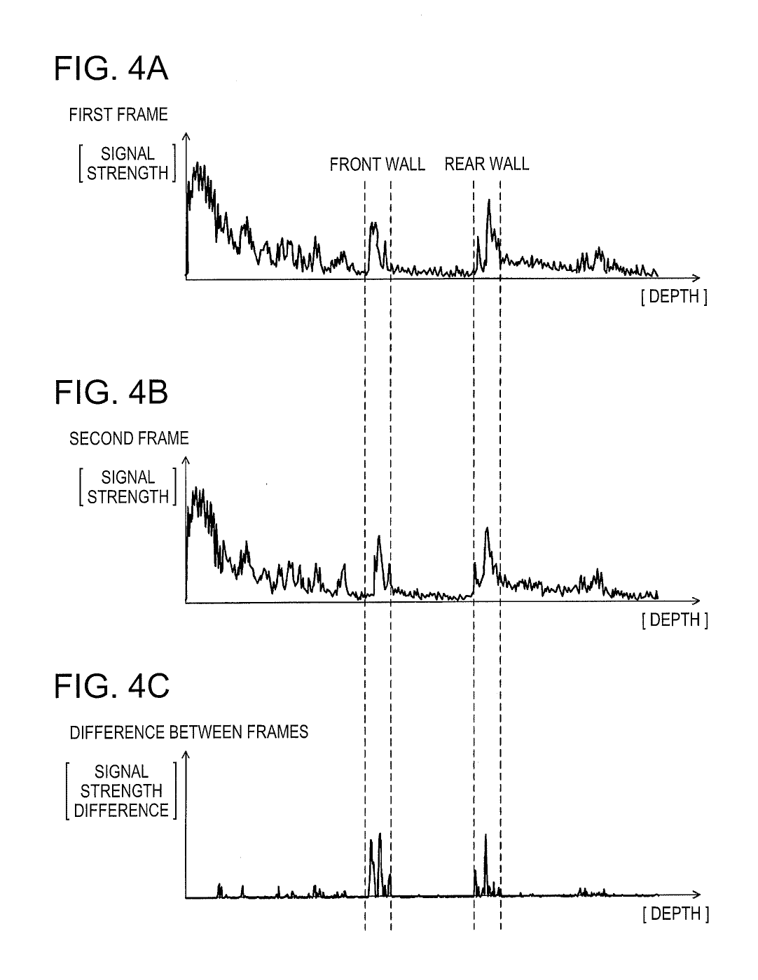

FIGS. 4A to 4C are diagrams showing an example of the received signal of the reflected wave at the position of the ultrasonic transducer 18 located immediately above the blood vessel. FIG. 4A is a “depth-signal strength graph” showing a measurement result in the first frame of the measurement period, and FIG. 4B is a “depth-signal strength graph” showing a measurement result in the second frame of the measurement period. FIG. 4C is a “graph of the signal strength difference between frames” showing a difference in the “depth-signal strength graph” between the first and second frames.

As described above, if there is the blood vessel 4, a strong reflected wave relevant to the front and rear walls is detected. Also in FIGS. 4A and 4B, peaks of two strong reflected waves that can be clearly identified appear at positions deeper than the group of reflected waves near the body surface. By calculating the signal strength difference between the first and second frames for each depth, the graph shown in FIG. 4C is obtained. Therefore, the movement of the front and rear walls of the blood vessel become clear between frames.

As is apparent from the graph in FIG. 4C, a slight signal strength difference occurs because body tissues other than the blood vessel are also slightly moved due to the influence of pulsation or the like. However, a large value as the value for the blood vessel (specifically, front and rear walls of the blood vessel) is not detected. Even more, such a peak is not seen in the signal strength difference graph of the reflected wave signal in the ultrasonic transducer 18 that is not located immediately above the blood vessel. That is, it can be said that the movement of the blood vessel due to pulsation appears in a change in the signal strength between frames having a time difference therebetween.

In the present embodiment, even if a change in the signal strength appropriate to the movement of the blood vessel is measured, it is not determined immediately that the ultrasonic transducer 18 is located immediately above the blood vessel, and the determination is made by statistically processing the change in the signal strength.

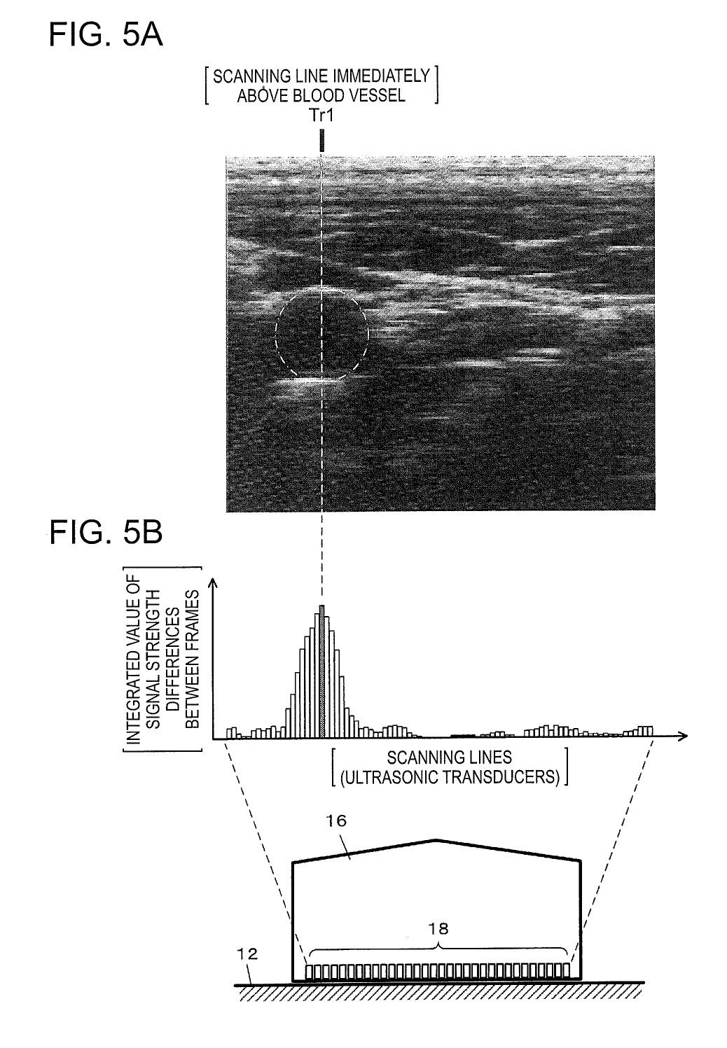

FIGS. 5A and 5B are diagrams for explaining the statistical processing on the change in the signal strength between two consecutive frames. FIG. 5A is an image obtained by converting the signal strength of the reflected wave in each ultrasonic transducer 18 into a brightness, that is, a B-mode image. FIG. 5B is a histogram obtained by calculating the signal strength change in each ultrasonic transducer 18 between two consecutive frames multiple times and integrating the signal strength changes. The point to note herein is that the horizontal axis of the graph in FIG. 4C is a depth direction and the graph is based on the reception result of one ultrasonic transducer 18, while the horizontal axis of the graph in FIG. 5B indicates the arrangement order of ultrasonic transducers 18 (that is, a scanning direction and a direction along the body surface of the subject 2).

This will be specifically described. The histogram shown in FIG. 5B can be obtained by repeating calculation of the sum of the signal strength differences at all depths for each ultrasonic transducer 18 whenever ultrasonic measurement for two consecutive frames is performed and by integrating the sums of the signal strength differences for a predetermined amount of time (for example, at least one to several beats in a cardiac cycle; about several seconds). In other words, the histogram shown in FIG. 5B is a result of statistical processing in which temporal changes of the signal in the depth direction at the same position on the body surface are integrated (summed) to one point of the same position.

For the sum of the signal strength difference obtained from the ultrasonic measurement for two consecutive frames, the sum for the ultrasonic transducers 18 located on the blood vessel is a larger value than the sum for the ultrasonic transducers 18 that are not located on the blood vessel. In addition, the larger the number of ultrasonic transducers 18 located immediately above the blood vessel center, the larger the value. Needless to say, this also appears in the signal strength difference. Accordingly, the ultrasonic transducer 18 for which the value on the vertical axis of the histogram satisfies predetermined height change conditions can be determined to be an ultrasonic transducer located immediately above the blood vessel. More specifically, the ultrasonic transducer 18 corresponding to the peak of the value on the vertical axis of the histogram is determined to be an ultrasonic transducer located immediately above the blood vessel, that is, a scanning line immediately above the blood vessel. In the example shown in FIGS. 5A and 5B, an ultrasonic transducer Tr1 corresponds to this.

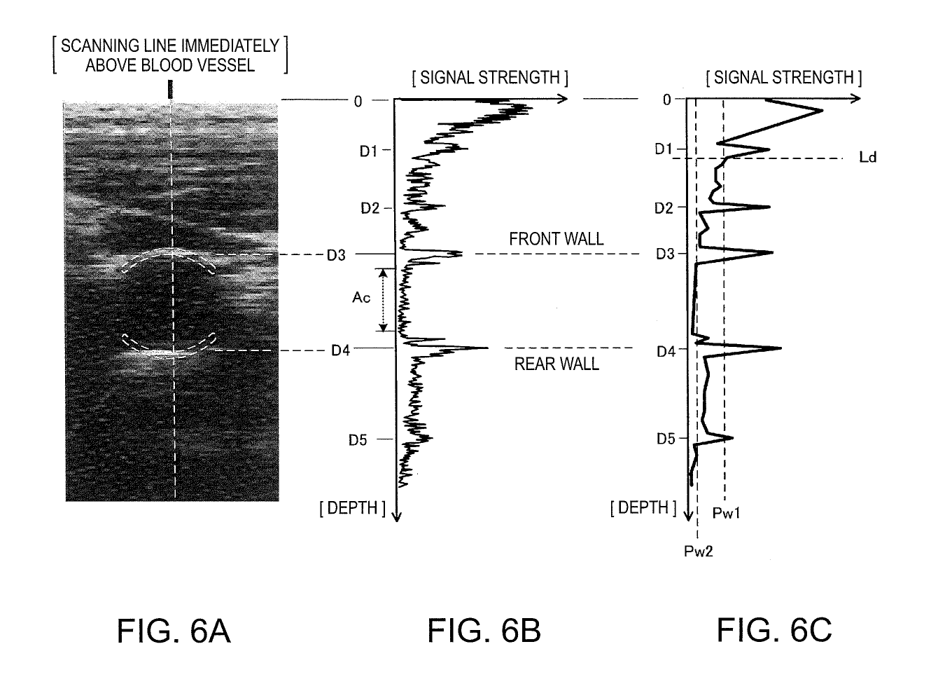

Next, a step of detecting a vessel wall depth position candidate (step S4 in FIG. 2) will be described. FIGS. 6A to 6C are diagrams for explaining the principle of the detection of a vessel wall depth position candidate. FIG. 6A is a B-mode image of a blood vessel part, FIG. 6B is a signal strength graph of the received signal of the reflected wave in the scanning lines immediately above the blood vessel, and FIG. 6C is a graph obtained by smoothing changes in the signal strength more clearly.

First, peaks, at which signal strengths equal to or higher than a predetermined vessel wall equivalent signal level Pw1 are obtained, are extracted. In this case, a strong reflected wave equal to or higher than the vessel wall equivalent signal level Pw1 is obtained from the front and rear walls of the blood vessel, but a strong reflected wave may also be similarly obtained from the surrounding tissues. For this reason, a plurality of peaks (in FIGS. 6A to 6C, five peaks D1 to D5) may appear in the signal strength graph. Therefore, the peaks are narrowed down based on the likelihood of the vascular wall.

In the narrowing down, first, a peak of a shallower position than the minimum reference depth Ld is excluded from the plurality of peaks D1 to D5. The minimum reference depth Ld is the limit of shallowness at which a blood vessel having an appropriate size as a measurement target can be present, and a value deeper than at least the dermis is set as the minimum reference depth Ld. In the example shown in FIGS. 6A to 6C, the peak D1 is excluded from the vessel wall depth position candidates since the depth of the peak D1 is less than the minimum reference depth Ld.

Then, the peaks are narrowed down based on the finding that the signal strength of the reflected wave of the intravascular lumen is very low compared with the surrounding tissues. That is, the peaks of the signal strength regarded as the vessel wall depth position candidates are determined as a pair of front and rear walls, and are temporarily combined. Then, the signal strengths between the respective combinations are statistically processed to calculate an average value or a median. Then, a combination satisfying the vascular front and rear walls pair equivalent conditions of “combination in which the statistical processing value is less than a predetermined intravascular lumen equivalent signal level Pw2” and “combination in which another peak is not present between the combined peaks” is extracted, and this is set as a “front and rear walls pair”.

For example, in FIG. 6C, a combination in which the peak D4 is regarded as the front wall and the peak D5 is regarded as the rear wall is excluded since the statistical processing value of the signal strength between the two peaks exceeds the intravascular lumen equivalent signal level Pw2. In addition, a combination in which the peak D3 is regarded as the front wall and the peak D5 is regarded as the rear wall and a combination in which the peak D2 is regarded as the front wall and the peak D4 is regarded as the rear wall are also excluded since another peak is present between these peaks. On the other hand, a combination in which the peak D3 is regarded as the front wall and the peak D4 is regarded as the rear wall satisfies the conditions described above. Accordingly, this combination is regarded as a “front and rear walls pair”.

As a method of narrowing down, focusing on the finding that the vascular wall shows a larger movement than the surrounding tissues, determination may be made from the displacement in one cardiac cycle of the peak position of the signal strength difference between frames. In the narrowing down method, however, for example, in a situation where there is almost no movement at the position of the front wall or the rear wall of the blood vessel in the positional relationship between the blood vessel 4 and the surrounding tissues, it is not possible to correctly narrow down the vascular front and rear walls pairs. However, according to the narrowing down method of the present embodiment, it is possible to reliably identify the vascular front and rear walls pair even in such a situation.

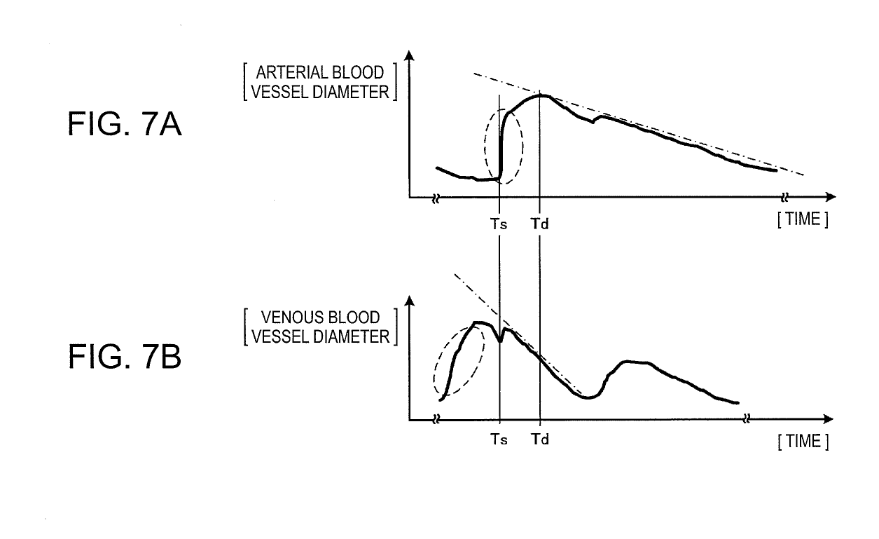

Next, an artery determination step (step S8 in FIG. 2) will be described. FIGS. 7A and 7B show waveforms of a change in the blood vessel diameter for approximately one beat of the cardiac cycle. FIG. 7A is a waveform of the arterial blood vessel diameter, and FIG. 7B is a waveform of the venous blood vessel diameter.

The vascular wall of the artery has a structure with high stretchability and elasticity so as to be able to withstand a pulsatile blood flow, which flows from the heart, and the blood pressure. For this reason, according to the beating of the heart, the blood vessel diameter increases rapidly during systole (Ts) and decreases slowly during diastole (Td) to return to the original thickness. Therefore, since the blood vessel diameter increases rapidly immediately after systole (Ts), the graph of the arterial blood vessel diameter rises abruptly (for example, a portion surrounded by the dashed line in FIG. 7A). On the other hand, since the blood vessel diameter decreases slowly during diastole (Td), the graph falls gently. Thus, in the case of the artery, the degree of change in a direction in which the blood vessel diameter increases is larger than that in a direction in which the blood vessel diameter decreases, and the difference is noticeable.

On the other hand, the vascular wall (vein wall) of the vein is thinner than the vascular wall (artery wall) of the artery. For this reason, the vascular wall (vein wall) of the vein has poor elasticity. In addition, blood pressure applied to the vein wall is lower than the blood pressure applied to the artery wall. Therefore, in the case of the vein, when the degree of change in the rise (a portion surrounded by the dashed line in FIG. 7B) of the graph in a direction in which the blood vessel diameter increases is compared with the degree of change in the lowering of the graph in which the blood vessel diameter decreases, the difference as in the case of the artery does not appear.

In the present embodiment, the difference in the degree of change in the blood vessel diameter due to pulsation of the artery and the vein is used for artery determination. Specifically, a temporal change in the distance between the front and rear walls, that is, the rate of change in the blood vessel diameter (hereinafter, referred to as a “diameter change rate”) is calculated by setting the position of the vascular wall (front and rear walls) regarded as the vascular front and rear walls pair as a region of interest and calculating the displacement rate of the vascular wall from the amount of displacement per unit time using the tracking function for tracking each region of interest between different frames.

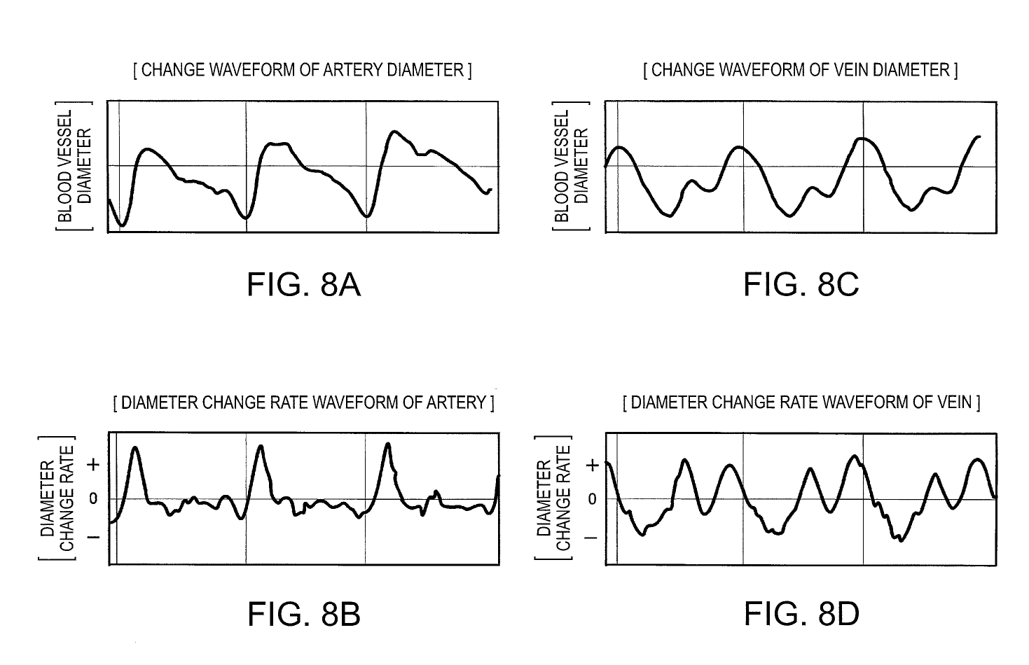

FIGS. 8A to 8D show waveforms of a change in the blood vessel diameter for approximately three beats of the cardiac cycle and waveforms of the diameter change rate corresponding to the change in the blood vessel diameter. FIGS. 8A and 8B are waveforms for the artery, and FIGS. 8C and 8D are waveforms for the vein. For the diameter change rate, a change in a direction in which the blood vessel diameter increases is “positive (+)”, and a change in a direction in which the blood vessel diameter decreases is “negative (−)”.

A blood vessel repeats periodic expansion and contraction with the cardiac cycle as a unit. That is, a period of one cardiac beat is divided into a diastole in which the blood vessel diameter increases to expand the blood vessel and a systole in which the blood vessel diameter decreases to contract the blood vessel. Whether the period of one cardiac beat is a diastole or a systole is determined from the blood vessel diameter change rate. That is, it is assumed that the period of one cardiac beat is a diastole if the diameter change rate is “positive” and is a systole if the diameter change rate is “negative”. The point to note herein is that the diastole and the systole are defined based on the contraction of the blood vessel instead of the contraction of the heart.

As shown in FIGS. 7A and 7B, there is a large difference in the degree of change in a direction in which the blood vessel diameter increases between the artery and the vein. That is, in the artery, the blood vessel diameter increases rapidly to expand the blood vessel. Accordingly, the degree of change in a direction of increase is large. On the other hand, in the vein, the blood vessel diameter increases gradually. Accordingly, the degree of change in a direction of increase is small compared with that in the case of the artery. This difference appears as a difference in the time length of the diastole.

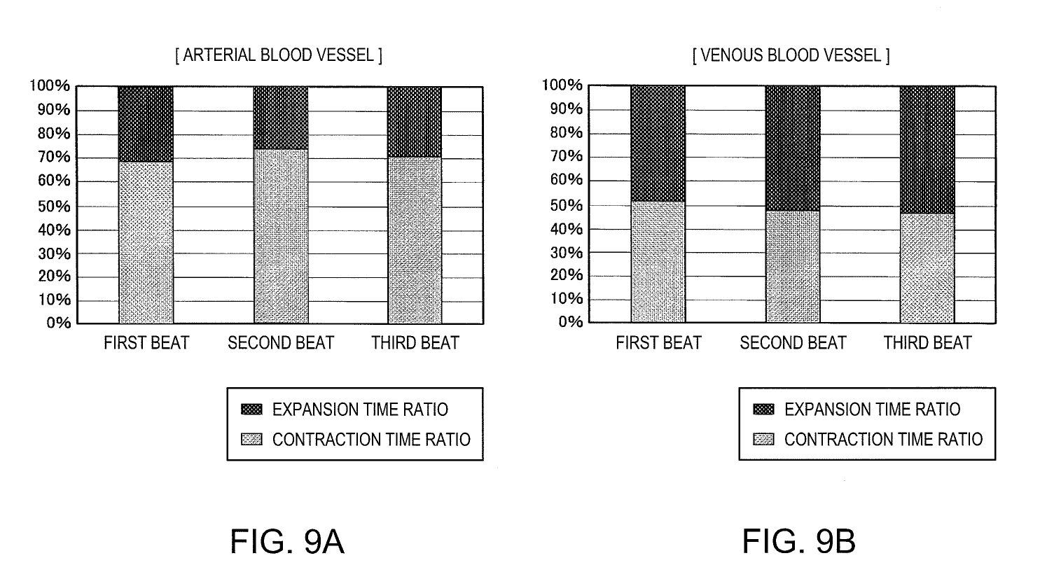

FIGS. 9A and 9B are bar graphs showing the ratio between the length of diastolic time (expansion time) and the length of systolic time (contraction time) per period of one cardiac beat that are obtained from the waveforms of the blood vessel diameter change rate shown in FIGS. 8A to 8D. FIG. 9A is a graph of an artery, and FIG. 9B is a graph of a vein.

As shown in FIG. 9, a significant difference in the ratio between the expansion time and the contraction time in a period of one beat of a cardiac cycle is observed. That is, in the case of the artery, the degree of change in a direction in which the blood vessel diameter increases is large (fast) compared with the degree of change in a direction in which the blood vessel diameter decreases. Accordingly, the contraction time is longer than the expansion time. The contraction time is about two to three times, for example, 2.3 times the expansion time. On the other hand, in the case of the vein, the degree of change in a direction in which the blood vessel diameter increases is almost the same as the degree of change in a direction in which the blood vessel diameter decreases. Accordingly, the expansion time and the contraction time are almost the same.

In the present embodiment, the ratio (=contraction time/expansion time) of expansion time to contraction time of the blood vessel diameter in a period of one cardiac beat is defined as an expansion contraction time ratio. From the expansion contraction time ratio, it is determined whether the blood vessel is an artery or a vein. “About 2.3” that is the expansion contraction time ratio in the artery shown as an example in FIG. 9A is almost the same value even though there are some differences depending on the age, sex, medical history, or the like of the subject that is assumed. Accordingly, a value lower than “about 2.3”, for example, “2.0” is set to a threshold value of conditions that the expansion contraction time ratio can have when the blood vessel is an artery, and it is determined that the blood vessel is an artery if the expansion contraction time ratio is equal to or greater than the threshold value and is a vein if the expansion contraction time ratio is less than the threshold value. In addition, the setting of a threshold value can be appropriately changed. For example, since the expansion contraction time ratio of the vein is a value close to “1.0”, the threshold value may be set to about “1.5”, and it may be determined that the blood vessel is an artery if the expansion contraction time ratio is equal to or greater than the threshold value and is a vein if the expansion contraction time ratio is less than the threshold value.

### Functional Configuration

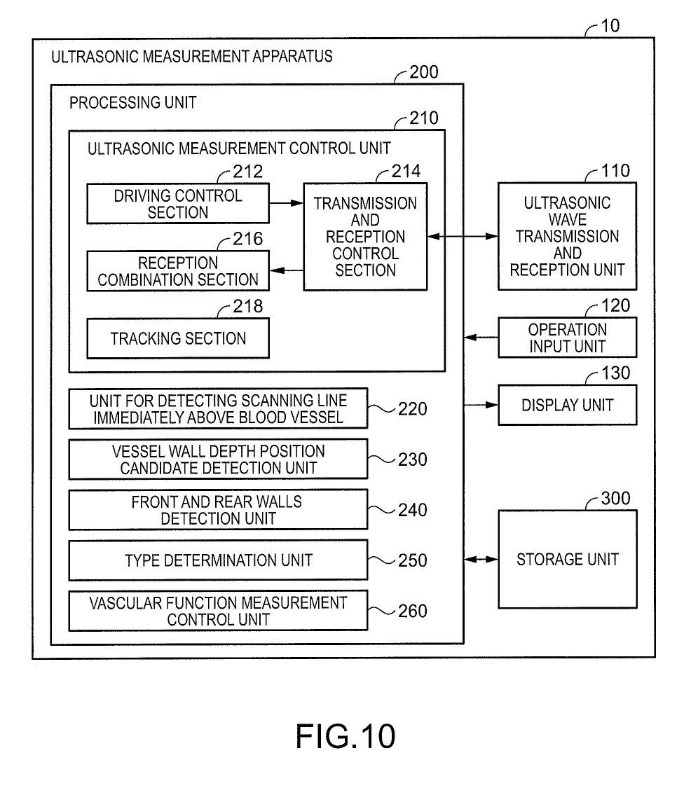

FIG. 10 is a diagram showing the functional configuration of the ultrasonic measurement apparatus 10. As shown in FIG. 10, the ultrasonic measurement apparatus 10 includes an ultrasonic wave transmission and reception unit 110, an operation input unit 120, a display unit 130, a processing unit 200, and a storage unit 300.

The ultrasonic wave transmission and reception unit 110 transmits an ultrasonic wave with a pulse voltage output from the processing unit 200. Then, the ultrasonic wave transmission and reception unit 110 receives a reflected wave of the transmitted ultrasonic wave, converts the reflected wave into a reflected wave signal, and outputs the reflected wave signal to the processing unit 200. In FIG. 1, the ultrasonic probe 16 corresponds to the ultrasonic wave transmission and reception unit 110.

The operation input unit 120 receives various kinds of operation input by the operator, and outputs an operation input signal corresponding to the operation input to the processing unit 200. This operation input unit 120 is realized by an input device, such as button switches, a touch panel, or various sensors. In FIG. 1, the touch panel 12 or the keyboard 14 corresponds to the operation input unit 120.

The display unit 130 is realized by a display device, such as a liquid crystal display (LCD), and performs various kinds of display based on the display signal from the processing unit 200. In FIG. 1, the touch panel 12 corresponds to the display unit 130.

The processing unit 200 is realized by a microprocessor such as a central processing unit (CPU) or a graphics processing unit (GPU), an application specific integrated circuit (ASIC), or an electronic component such as an integrated circuit (IC) memory, and controls the operation of the ultrasonic measurement apparatus 10 by performing various kinds of arithmetic processing based on a program or data stored in the storage unit 300, an operation signal from the operation input unit 110, and the like. In FIG. 1, the CPU 32 mounted on the control board 31 corresponds to the processing unit 200. The processing unit 200 includes an ultrasonic measurement control unit 210, a unit for detecting a scanning line immediately above a blood vessel 220, a vessel wall depth position candidate detection unit 230, a front and rear walls detection unit 240, a type determination unit 260, and a vascular function measurement control unit 270.

The ultrasonic measurement control unit 210 includes a driving control section 212, a transmission and reception control section 214, a reception combination section 216, and a tracking section 218, and controls the transmission and reception of the ultrasonic wave in the ultrasonic wave transmission and reception unit 110.

The driving control section 212 controls the transmission timing of ultrasonic pulses from the ultrasonic wave transmission and reception unit 110, and outputs a transmission control signal to the transmission and reception control section 214.

The transmission and reception control section 214 generates a pulse voltage according to the transmission control signal from the driving control section 212, and outputs the pulse voltage to the ultrasonic wave transmission and reception unit 110. In this case, it is possible to adjust the output timing of the pulse voltage to each ultrasonic transducer by performing transmission delay processing. In addition, the transmission and reception control section 214 performs the amplification or filtering of the reflected wave signal input from the ultrasonic wave transmission and reception unit 110, and outputs the result to the reception combination section 216.

The reception combination section 216 generates reflected wave data 320 by performing delay processing as necessary, that is, by performing various kinds of processing relevant to the so-called focus of a received signal.

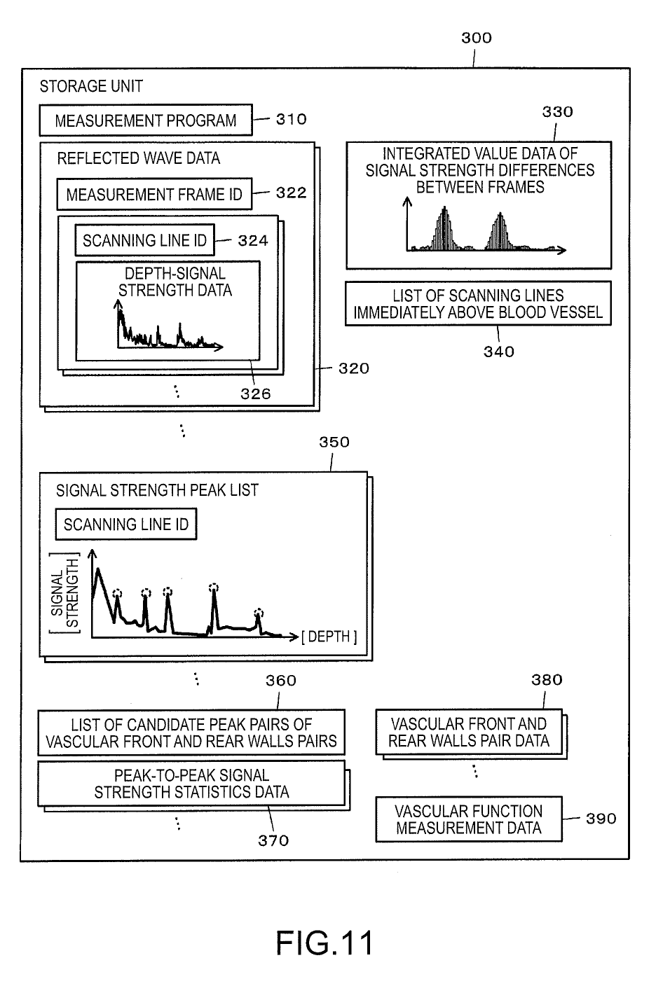

As shown in FIG. 11, the reflected wave data 320 is generated for each frame. A piece of reflected wave data 320 includes a corresponding measurement frame ID 322, scanning line ID 324, and depth-signal strength data 326 corresponding thereto.

The tracking section 218 performs processing relevant to so-called “tracking” that is for tracking the position of a region of interest between frames of ultrasonic measurement based on the reflected wave data (reflected wave signal). For example, it is possible to perform processing for setting a region of interest (tracking point) in the reflected wave data (for example, a B-mode image) as a reference, processing for tracking each region of interest between different frames, and processing for calculating the displacement for each region of interest. Thus, known functions, such as “phase difference tracking” or “echo tracking” are realized.

The unit for detecting a scanning line immediately above a blood vessel 220 performs arithmetic processing for detecting the scanning lines immediately above the blood vessel or controls each unit. That is, control relevant to the above-described step of detecting the scanning lines immediately above the blood vessel is performed (refer to FIGS. 3 to 5B). In the detection of a scanning line immediately above the blood vessel, the calculation of the sum of the signal strength difference between two frames at all depths is repeated for each ultrasonic transducer whenever ultrasonic measurement for two consecutive frames is performed to generate the reflected wave data 320, and the signal strength difference is integrated as integrated value data of signal strength differences between frames 330 for a predetermined amount of time. Then, the ultrasonic transducer (scanning line) having an integrated value that satisfies predetermined height change conditions is detected as a scanning line immediately above the blood vessel. The scanning line immediately above the blood vessel and the detected scanning line ID are stored as a list of scanning lines immediately above a blood vessel 340.

The vessel wall depth position candidate detection unit 230 detects a depth position regarded as a vessel wall based on the received signal of the reflected wave in the scanning lines immediately above the blood vessel. That is, a part of control relevant to the above-described step of detecting the vessel wall depth position candidate is performed (refer to FIG. 6A). In the detection of a vessel wall depth position candidate, a depth position candidate regarded as a vascular wall, that is, a peak of the signal strength, is extracted from the depth-signal strength data 326 of the scanning line for each scanning line immediately above the blood vessel, thereby generating a signal strength peak list 350.

The front and rear walls detection unit 240 detects the front and rear walls of the blood vessel using the received signal in the scanning lines immediately above the blood vessel. That is, a part of control relevant to the above-described step of narrowing down the front and rear walls pair of the blood vessel is performed (refer to FIG. 6C). In the detection of front and rear walls of the blood vessel, a combination of the peak assumed to be a front wall and the peak assumed to be a rear wall is generated from the peaks of the signal strength stored in the signal strength peak list 350, that is, from the depth position candidates regarded as vascular walls, and this is stored as the list of candidate peak pairs of vascular front and rear walls pairs 360. Then, a statistical value of the signal strength between the peaks of the pair is calculated for each pair of peaks assumed to be front and rear walls that has been generated, and this is stored as peak-to-peak signal strength statistics data 370. In addition, for each pair of peaks, a pair in which the statistical value of the signal strength between the peaks of the pair satisfies the vascular front and rear walls pair equivalent conditions is narrowed down, and the pair is detected as a “front and rear walls pair”.

A contraction and expansion time calculation unit 250 calculates the contraction time and the expansion time of a blood vessel using a temporal change in the distance between the front and rear walls. That is, a part of control relevant to the artery determination step described above is performed (refer to FIGS. 7A to 8D). In the calculation of the contraction time and expansion time, front and rear walls are set as regions of interest for each vascular front and rear walls pair, and the displacement of each frame is acquired by tracking over a predetermined period (for example, ten beats or more of the cardiac cycle). Then, for each frame, a relative speed V (=Vf−Vr) between the displacement speed Vf of the front wall and the displacement speed Vr of the rear wall is set as a change in the distance between the front and rear walls, that is, a blood vessel diameter change rate, and it is determined whether the frame is a diastole or a systole according to the sign (positive or negative) of the blood vessel diameter change rate. Then, the number of frames determined to be a diastole is set as an expansion time, and the number of frames determined to be a systole is set as a contraction time.

A type determination unit 260 determines the type (artery or vein) of a blood vessel using the relative relationship between the expansion time and the contraction time of the blood vessel. That is, a part of control relevant to the artery determination step described above is performed (refer to FIGS. 7A to 9B). In the type determination, it is determined whether the blood vessel is an artery or a vein by comparing the expansion contraction time ratio, which is a ratio between the number of frames determined to be a diastole and the number of frames determined to be a systole (the number of frames of a systole/the number of frames of a diastole), with a predetermined threshold value.

The vascular function measurement control unit 270 performs control relevant to predetermined vascular function measurement by continuing position measurement with the front and rear walls of the blood vessel determined to be an artery by the type determination unit 260 as a tracking target.

The storage unit 300 is realized by a storage device, such as a ROM, a RAM, or a hard disk, and stores a program or data required for the processing unit 200 to perform overall control of the ultrasonic measurement apparatus 10. In addition, the storage unit 300 is used as a working area of the processing unit 200, and temporarily stores calculation results of the processing unit 200, operation data from the operation input unit 120, and the like. In FIG. 1, the storage medium 33 mounted on the control board 31 corresponds to the storage unit 300. In the present embodiment, a measurement program 310, the reflected wave data 320, the integrated value data of signal strength differences between frames 330, the list of scanning lines immediately above a blood vessel 340, the signal strength peak list 350, the list of candidate peak pairs of vascular front and rear walls pairs 360, the peak-to-peak signal strength statistics data 370, vascular front and rear walls pair data 380, and vascular function measurement data 390 are stored in the storage unit 300.

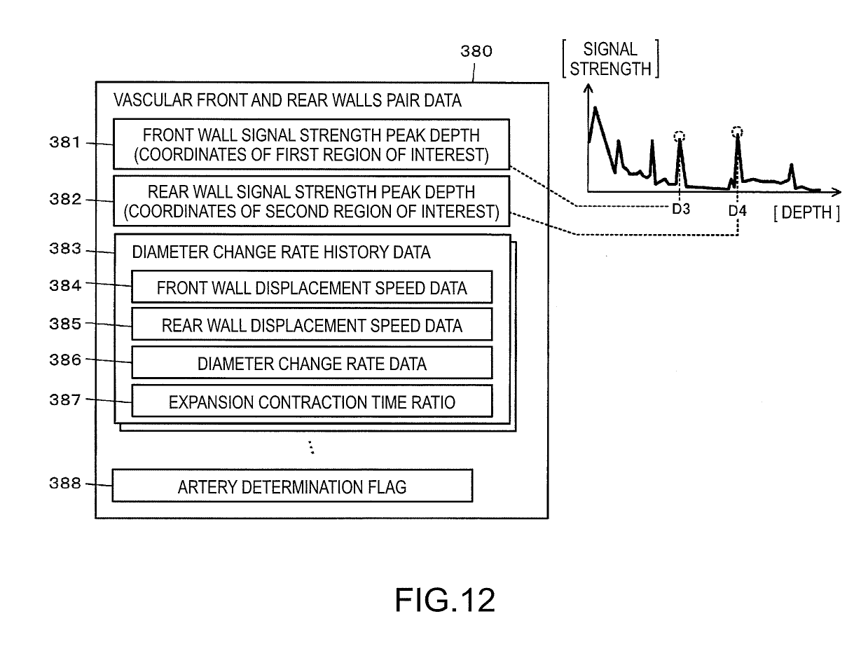

FIG. 12 is a diagram showing the data configuration of the vascular front and rear walls pair data 380. The vascular front and rear walls pair data 380 is generated for each vascular front and rear walls pair, and includes a front wall signal strength peak depth 381, a rear wall signal strength peak depth 382, diameter change rate history data 383, and an artery determination flag 388.

The front wall signal strength peak depth 381 and the rear wall signal strength peak depth 382 are depth positions of the peaks of the signal strengths regarded as front and rear walls, and correspond to the coordinates of a first region of interest and the coordinates of a second region of interest in the tracking control for artery determination, respectively. The diameter change rate history data 383 is generated for each period of one cardiac beat, and includes front wall displacement speed data 384, rear wall displacement speed data 385, blood vessel diameter change rate data 386, and expansion contraction time ratio 387 in the period of one cardiac beat. The front wall displacement speed data 384 and the rear wall displacement speed data 385 are time-series data of the displacement of each of the front and rear walls acquired by tracking. The blood vessel diameter change rate data 386 is time-series data of a change in the distance between the front and rear walls calculated from the front wall displacement speed data 384 and the rear wall displacement speed data 385, that is, time-series data of the blood vessel diameter change rate. The artery determination flag 388 is a flag for storing a determination result regarding whether or not the blood vessel is an artery, and “1” is set when it is determined that the blood vessel is an artery.

### Flow of Process

Next, the operation of the ultrasonic measurement apparatus 10 in each step from the detection of the scanning lines immediately above the blood vessel to artery determination will be described (refer to FIG. 2).

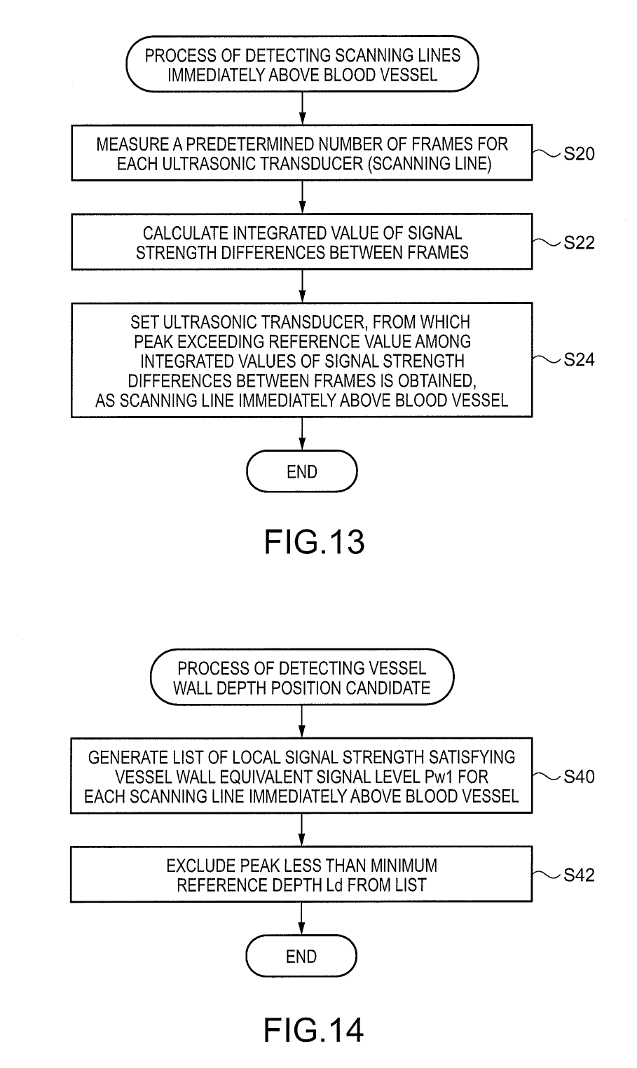

FIG. 13 is a flowchart illustrating the flow of the process of detecting the scanning lines immediately above the blood vessel. Referring to FIG. 13, the unit for detecting a scanning line immediately above a blood vessel 220 transmits ultrasonic beams of a predetermined number of frames to each ultrasonic transducer (scanning line) provided in the ultrasonic wave transmission and reception unit 110 and receives the reflected waves (step S20). Accordingly, the reflected wave data 320 is stored in the storage unit 300.

Then, signal strength differences between consecutive frames at all depths are calculated from the reflected wave data 320 for each ultrasonic transducer, and the integrated value data of signal strength differences between frames 330 is calculated by integrating the signal strength differences (step S22). Then, an ultrasonic transducer from which a peak exceeding a predetermined reference value is obtained is determined to be the scanning line immediately above the blood vessel, and the scanning line ID corresponding to the ultrasonic transducer is registered in the list of scanning lines immediately above a blood vessel 340 (step S24). Then, the process of detecting the scanning lines immediately above the blood vessel is ended.

FIG. 14 is a flowchart illustrating the flow of the process of detecting the vessel wall depth position candidate. Referring to FIG. 14, the vessel wall depth position candidate detection unit 230 extracts a local peak, at which the signal strength satisfies the predetermined vessel wall equivalent signal level Pw1 (refer to FIG. 6C), from the reflected wave data 320 of the scanning line for each scanning line immediately above the blood vessel that is registered in the list of scanning lines immediately above a blood vessel 340, thereby generating the signal strength peak list 350 (step S40). Then, peaks of the signal strength equal to or less than the minimum reference depth Ld are excluded from the list (step S42), and the process of detecting the vessel wall depth position candidate is ended.

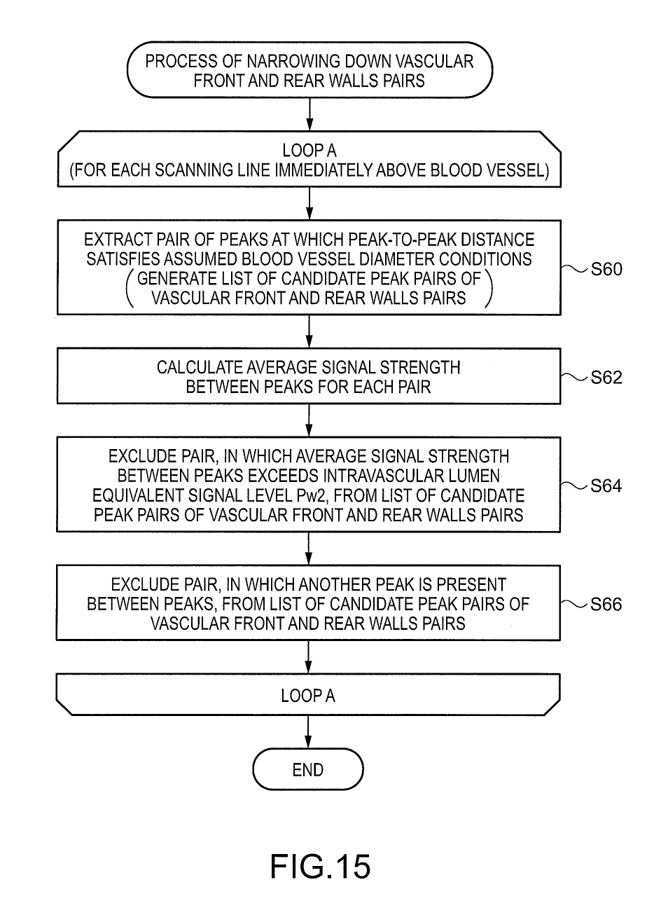

FIG. 15 is a flowchart illustrating the process of narrowing down vascular front and rear walls pairs. Refer to FIG. 15, the front and rear walls detection unit 240 executes a loop A for each scanning line immediately above the blood vessel that is registered in the list of scanning lines immediately above a blood vessel 340 (steps S60 to S66).

In the loop A, a pair is generated from the registered peaks with reference to the signal strength peak list 350 corresponding to the scanning lines immediately above the blood vessel to be processed, and a pair in which a peak-to-peak distance satisfies predetermined assumed blood vessel diameter conditions is extracted, thereby generating the list of candidate peak pairs of vascular front and rear walls pairs 360 (step S60). The assumed blood vessel diameter conditions referred to herein are conditions defining a rough range of the blood vessel diameter suitable for the measurement, and it is assumed that the assumed blood vessel diameter conditions are set in advance by tests or the like.

Then, an average signal strength between peaks is calculated for each pair of peaks registered in the list of candidate peak pairs of vascular front and rear walls pairs 360 (step S62), and a pair in which the average signal strength between peaks exceeds the intravascular lumen equivalent signal level Pw2 (refer to FIG. 6C) is excluded from the list of candidate peak pairs of vascular front and rear walls pairs 360 (step S64). Among the peaks registered in the list of candidate peak pairs of vascular front and rear walls pairs 360, a pair in which another peak is present between peaks is excluded from the list (step S66), and the loop A is ended. The pair of peaks remaining in the list of candidate peak pairs of vascular front and rear walls pairs 360 in this stage is front and rear walls of the blood vessel in the scanning lines immediately above the blood vessel to be processed.

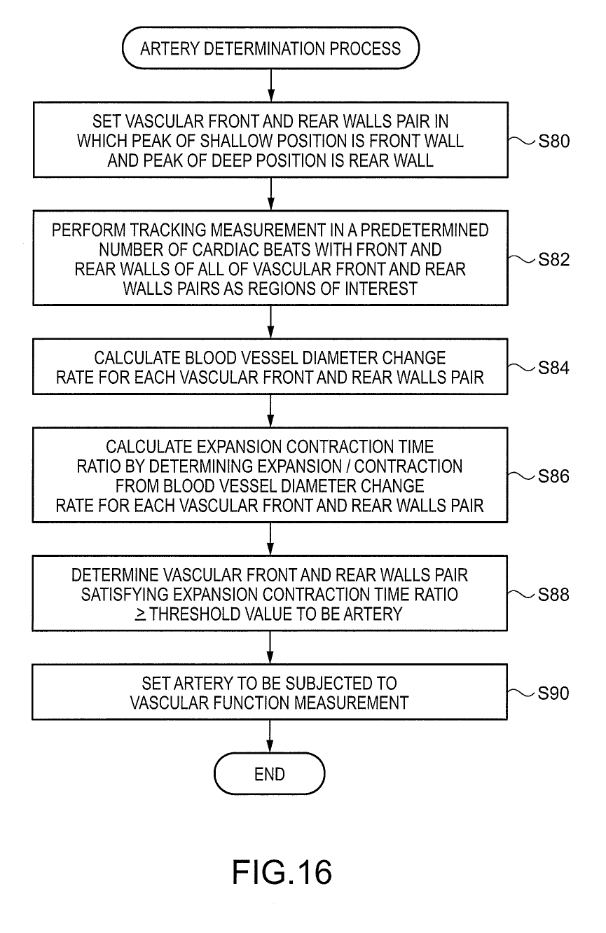

FIG. 16 is a flowchart illustrating the flow of the artery determination process. Referring to FIG. 16, the contraction and expansion time calculation unit 250 sets a vascular front and rear walls pair by regarding the peak of a relatively shallow position as a front wall and the peak of a relatively deep position as a rear wall for each of the peak pairs registered in the list of candidate peak pairs of vascular front and rear walls pairs 360 (step S80). Then, the front and rear walls of each vascular front and rear wall pair are set as regions of interest, and tracking of each region of interest is performed for a predetermined amount of time (a period of a predetermined number of beats of a cardiac cycle) (step S82).

Then, for each vascular front and rear walls pair, time-series data of the blood vessel diameter change rate is calculated from the time-series data of the displacement of each of the front and rear walls acquired by tracking (step S84). By determining a diastole/systole from the sign (positive or negative) of the diameter change rate, an expansion time and a contraction time are calculated. Then, the type determination unit 260 calculates the expansion contraction time ratio that is a ratio between the calculated expansion time and the calculated contraction time (step S86). Then, a vascular front and rear walls pair having an expansion contraction time ratio equal to or greater than a predetermined threshold value, among the vascular front and rear walls pairs, is determined to be an artery (step S88), and a blood vessel (artery) to be subjected to vascular function measurement among the blood vessels determined to be arteries is set (step S90). Then, the artery determination process is ended.

### Effects

As described above, according to the ultrasonic measurement apparatus 10 of the present embodiment, it is possible to find an artery automatically from the body tissues in the scanning range of the ultrasonic probe 16 and to perform vascular function measurement with the artery as a measurement target. Therefore, since the only thing that the operator has to do is to place the ultrasonic probe 16 at an approximate place where the carotid artery may be present, labor in the measurement work is greatly reduced. As a result, measurement errors can also be significantly reduced.

In addition, it should be understood that embodiments to which the invention can be applied is not limited to the embodiment described above and various modifications can be made without departing from the spirit and scope of the invention.

The entire disclosure of Japanese Patent Application No. 2014-038977, filed on Feb. 28, 2014 is expressly incorporated by reference herein.

What is claimed is:

1. An ultrasonic measurement apparatus, comprising:
a transmission and reception control unit that controls transmission of an ultrasonic wave to a blood vessel and reception of a reflected wave;
a contraction and expansion time calculation unit that calculates a contraction time and an expansion time of the blood vessel based on a received signal of the reflected wave; and
a type determination unit that determines a type of the blood vessel using the contraction time and the expansion time.

2. The ultrasonic measurement apparatus according to claim 1,
wherein the type determination unit determines the type of the blood vessel using a ratio between the contraction time and the expansion time.

3. The ultrasonic measurement apparatus according to claim 1,
wherein the type determination unit determines an artery and a vein as the type of the blood vessel.

4. The ultrasonic measurement apparatus according to claim 1,
wherein the type determination unit determines that the blood vessel is an artery using at least a ratio between the contraction time and the expansion time when the blood vessel is an artery.

5. The ultrasonic measurement apparatus according to claim 1,
wherein the type determination unit determines that the blood vessel is a vein using at least a ratio between the contraction time and the expansion time when the blood vessel is a vein.

6. The ultrasonic measurement apparatus according to claim 1, further comprising:
a front and rear walls detection unit that detects a front wall and a rear wall of the blood vessel using the received signal of the reflected wave,
wherein the contraction and expansion time calculation unit calculates the contraction time and the expansion time by determining a systole and a diastole of the blood vessel from a temporal change in the front and rear walls.

7. The ultrasonic measurement apparatus according to claim 1,
wherein the contraction and expansion time calculation unit calculates the contraction time and the expansion time using the received signal of a period of at least one cardiac beat.

8. An ultrasonic measurement method, comprising:
controlling transmission of an ultrasonic wave to a blood vessel and reception of a reflected wave;
calculating a contraction time and an expansion time of the blood vessel based on a received signal of the reflected wave; and
determining a type of the blood vessel using the contraction time and the expansion time.
